(19) United States
(12) Patent Application Publication
(10) Pub. No.: US 2025/0327888 A1
(43) Pub. Date: Oct. 23, 2025

(54) MRI WITH RF SHIELD FOR TRANSMIT COILS THAT REDUCES ACOUSTIC NOICE AND INCREASES TRANSMISSION OF RF ENERGY TO THE IMAGING VOLUME

(71) Applicant: MR CoilTech Limited, Glasgow (GB)

(72) Inventors: David A. FEINBERG, Sebastopol, CA (US); Shajan GUNAMONY, Glasgow (GB)

(21) Appl. No.: 18/638,215

(22) Filed: Apr. 17, 2024

(51) Int. Cl. G01R 33/42; A61B 5/055

(52) U.S. Cl. CPC G01R 33/42; A61B 5/055

(57) ABSTRACT

An MRI system includes an RF shield that reduces acoustic noise and increases transmission of RF energy from RF transmit coil to the imaging volume compared with conventional double-layer shields. The RF shield comprises strips of electrically conductive material that are elongated in the direction of the main magnetic field and are spaced from each other in the circumferential direction. The strips are bridged with capacitors. The RF shield reduces acoustic noise and eddy currents and increases transmission of RF energy compared with conventional shields of overlapping strips in two radially spaced layers.

### FIELD

This patent specification relates to Magnetic Resonance Imaging (MRI) and to reducing acoustic noise and improving RF transmit efficiency with a novel radio frequency (RF) shield for MRI transmit coils.

### BACKGROUND

Acoustic noise from magnetic resonance imaging (MRI) can cause patient anxiety and discomfort, hearing damage, and impaired communication between patients and health-care workers. This noise is generated by interactions between the static magnetic field and alternating current in gradient coils, resulting in Lorentz forces that vibrate the coils, creating mechanical noise. Lorentz forces increase with magnetic field strength and electrical current in gradient coils. MRI systems using 1.5-Tesla (T) and 3 T magnetic fields as well as increasingly powerful gradient electronics exposes patients to sound pressure levels (SPL) as high as 130 decibels (dB SPL), nearing the maximum allowable SPL of 140 dB as set by the United States Food and Drug Administration (FDA).

Higher field strength MRI, such as 7 T MRI with optimized pulse sequences may improve image resolution including in regions such as the brain and inner ear but can produce even greater acoustic noise than 1.5 and 3 T scanners. Louder noises can result in temporary or permanent hearing loss. See for example https://www.ncbi.nlm.nih.gov/pmc/articles/PMC10506133/, incorporated herein by reference. Ear plugs provide a low-cost passive protection that can decrease observed noise, but cochlear function can be affected during MRI even with passive protection, and ear plugs alone may be insufficient. Special pulse sequences designed to reduce acoustic noise can help, but a need remains for further reduction and for continuing to use existing, well-known pulse sequences rather than to use or develop special quiet sequences.

To reduce acoustic noise, gradient coils typically are encased in vibration reducing material. RF shielding has been used in MRI systems to generate a known pattern of eddy currents that is not RF-lossy. See for example U.S. Pat. No. 5,406,204 discussing a shield of overlapping strips of copper strips. See also Lee et al., B J, Watkins R D, Chang C M, Levin C S, Low eddy current RF shielding enclosure designs for 3T MR applications, Jun. 6, 2017, https://doi.org/10.1002/mrm.26766. The patent and the publication cited in this paragraph are hereby incorporated by reference. A conventional RF shield comprises flexible, double-layered copper strip. Each layer is slotted to reduce eddy currents and the strips of the two layers are offset and overlap to form a high value capacitor that is believed to low impedance at high frequency. High acoustic noise is present when using such conventional RF shielding, especially in higher main magnetic field MRI systems.

The subject matter described or claimed in this patent specification is not limited to embodiments that solve any specific disadvantages or that operate only in environments such as those described above. Rather, the above background is only provided to illustrate one exemplary technology area where some embodiments described herein may be practiced.

### SUMMARY

As described in the initially presented claims but subject to amendments thereof in prosecuting this patent application, according to some embodiments a Magnetic Resonance Imaging (MRI) system (12) is configured to reduce acoustic noise when imaging a patient and increase transmit efficiency and comprises: a magnet (12a) configured to provide a main magnetic field, gradient coils (12b) configured to selectively provide varying gradient magnetic fields, and a radio frequency (RF) transmit coil (12c) configured to selectively transmit RF pulses to an imaging volume; and an acoustic noise reducing RF shield (14) adjacent the RF transmit coil and comprising longitudinal strips (16) of an electrically conductive material and capacitors (18) bridging the strips.

According to some embodiments, the MRI system can further comprise one or more of: (a) the strips comprise a Phosphor Bronze Mesh (PBM); (b) the strips are spaced from each other; (c) the capacitors bridge adjacent strips and provide low impedance paths; (d) the RF shield reduces eddy currents compared with an RF shield of overlapping strips of conductive material; (e) the RF shield increases transmission of RF energy from the RF transmit coils to the imaging volume compared with an RF shield of overlapping strips of conductive material; (f) the strips are elongated along the main magnetic field; (g) the RF shield is a single layer of said strips; (h). the strips are a Phosphor Bronze Mesh (PBM) with 380 mesh count per inch; (i) the RF shield comprises 16 strips that are 2 mm apart and adjacent strips are bridged with 1000 pF capacitors; and (j) the strips (16) are spaced from each other to provide unobstructed gaps for passage of RF energy from said RF transmit coils.

According to some embodiments, a Magnetic Resonance Imaging (MRI) system comprises: a magnet (12a) configured to provide a main magnetic field, gradient coils (12b) configured to provide gradient magnetic fields, and a radio frequency (RF) transmit coil (12c) configured to transmit RF pulses to an imaging volume; and an RF shield (14) that is adjacent the RF transmit coil and comprises longitudinal strips (16) of an electrically conductive material and capacitors (18) bridging the strips; wherein the strips are spaced from each other to leave gaps between adjacent strips facilitating increased transfer of RF energy from said RF transmit coils to said imaging volume compared with RF screens with overlapping strips of electrically conductive material.

According to some embodiments, the MRI system described in the immediately preceding paragraph further comprises one of more of: (a) the RF shield comprises at least 10 strips; (b) the strips are spaced from each other by at least 1 mm; (c) the strips are in a single layer surrounding the imaging volume; (d) the RF shield surrounds the RF transmit coils and the imaging volume.

According to some embodiments, a Magnetic Resonance Imaging (MRI) method comprises: transmitting radio frequency (RF) energy from RF transmit coils to an imaging volume that is selectively subjected to a main magnetic field and time-varying gradient magnetic fields; and shielding with an RF shied that is adjacent the RF transmit coils and reduces acoustic noise with strips of electrically conductive material that surround the imaging volume, are elongated along the main magnetic field and are spaced from each in a circumferential direction around the imaging volume, and are bridged with capacitors.

According to some embodiments, the method further comprise one or more of: (a) increasing transmission of RF energy from the RF transmit coils with said spaced-apart strips compared with an RF shield of overlapping strips of conductive material; (b) said shielding comprises arranging the strips in a single layer surrounding the imaging volume; and (c) said shielding comprises arranging at least 10 of said strips uniformly spaced from each other around the imaging volume.

### BRIEF DESCRIPTION OF THE DRAWINGS

To further clarify the above and other advantages and features of the subject matter of this patent specification, specific examples of embodiments thereof are illustrated in the appended drawings. It should be appreciated that these drawings depict only illustrative embodiments and are therefore not to be considered limiting of the scope of this patent specification or the appended claims. The subject matter hereof will be described and explained with additional specificity and detail using the accompanying drawings in which:

FIG. 1 illustrates a conventional double-layered RF shield of flexible PCB in panel A and illustrates a novel RF shield comprising strips bridged with capacitors in panel B, according to some embodiments.

FIG. 1A schematically illustrates main subsystems of an MRI system, according to some embodiments.

FIG. 1B lustrates three strips (not to scale) and bridging capacitors of a novel RF shield that comprises additional strips and capacitors, according to some embodiments.

FIG. 2 illustrates sound pressure level sensors attached near the left and right ears of a head-shaped phantom filled with tissue equivalent solution.

FIG. 3 shows B1 maps acquired in CP mode that demonstrate improved transmit efficiency of an example of a novel PBM RF shield with a mesh structure that allows wider strips to be used without increasing eddy currents, for a rough gain of 15% in B₁⁺ between the two shield configurations relative to a conventional RF shield, according to some embodiments.

FIG. 4 shows acoustic transfer functions (in linear scale) demonstrating substantial noise reduction relative to a standard RF shield for all three gradient axes using an example of a novel RF shield providing up to 10 dB noise reduction, with measurements performed on each axis separately at constant gradient amplitude of 2 mT/m, according to some embodiments.

FIG. 5 shows a comparison of acoustic noise level in EPI at different echo spacings when using a novel RF shield, demonstrating almost systematically lower noise levels, where the axis indicates the readout direction of the EPI sequence, according to some embodiments.

FIG. 6 illustrates a setup of accelerometers placed on an external surface of an RF coil (top left) or on a table (bottom left) to measure vibrations orthogonal to the surface; different support materials and sponge window seals are shown on the right.

FIG. 7 illustrates SPL measurements performed at the right ear of an anthropomorphic phantom with and without the RF coil, versus frequency and at 5 mT/m; in the presence of the RF coil, an increasing trend of acoustic noise versus frequency, consistent with eddy-currents, is visible.

FIG. 8 illustrates vibration measurements (top) on the RF coil and acoustic noise levels (down, linear scale) versus frequency and gradient axis; different colors on the top correspond to different sensors; reasonable correlation between the two can be observed, suggesting together with the other data that much if not most of the SPL originales from the vibration of the RF coil.

FIG. 9 illustrates vibration measurements on the RF coil versus frequency for the X gradient axis and with different supporting materials; little differences can be observed; the differences on sensor #3 are believed due to different heights of the coil affecting the contact of the accelerometer with the boreliner.

FIG. 10 illustrates SPL measurements performed at the right (top) and left (bottom) ear of a volunteer with and without sponge window seals.

### DETAILED DESCRIPTION

A detailed description of examples of preferred embodiments is provided below. While several embodiments are described, the new subject matter described in this patent specification is not limited to any one embodiment or combinations of embodiments described herein but instead encompasses numerous alternatives, modifications, and equivalents. In addition, while numerous specific details are set forth in the following description to provide a thorough understanding, some embodiments can be practiced without some or all these details. Moreover, for the purpose of clarity, certain technical material that is known in the related art has not been described in detail to avoid unnecessarily obscuring the new subject matter described herein. It should be clear that individual features of one or several of the specific embodiments described herein can be used in combination with features of other described embodiments or with other features.

Like reference numbers and designations in the various drawings indicate like elements. Further, the reference numbers of components that are like in structure and function have the same second and third digits. For conciseness, components that bear the same reference numbers of the same last two digits of reference numbers are described only in connection with the Figure that first refers to them and the description is not repeated in connection with subsequently discussed Figures.

An important aspect of the novel approach to noise reduction described in this patent specification relates to a discovery that eddy currents induced in the RF shield are a significant and possibly primary source of acoustic noise in MR systems such as a 7T scanner. Notable reduction in acoustic noise is achieved by replacing a conventional slotted double-layered RF shield with a segmented phosphor bronze mesh to reduce such eddy currents and acoustic noise and enhance RF transmission, according to embodiments described below.

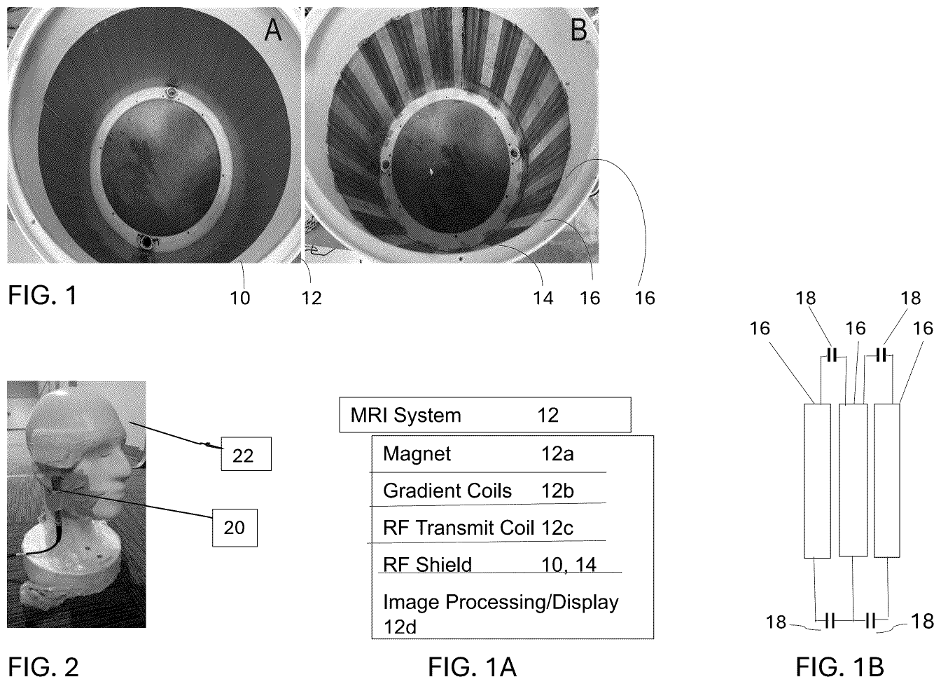

FIG. 1 illustrates a conventional double-layered RF shield 10 of flexible PCB in panel A as used in a current 7T MRI system 12 and illustrates in panel B an example of a novel RF shield 14 comprising strips 16 bridged with capacitors in the same MRI system. RF shields 10 and 14 surround the imaging volume inside the magnet of the MRI system.

FIG. 1A schematically illustrates main subsystems of an MRI system in which a novel RF shield 14 can replace a conventional RF shield 10.

FIG. 1B illustrates three of strips 16 and bridging capacitors 18 of RF shield 14. Measurements described below were performed using a 7T MRI system equipped with an investigational Impulse head gradient coil (Siemens Healthineers, Erlangen, Germany) capable of achieving a maximum gradient strength of 200 mT/m and slew rate of 900 T/m/s per axis. The changing magnetic flux from the time varying gradient magnetic field in such MRI systems induces eddy currents on any conductive surface. This translates into resistive heat and vibrations and unwanted acoustic noise, which the new approach described in this patent specification alleviates.

An example of RF shield 14 comprises PBM of 380 mesh count per inch (Shandong Xingying Technology, China), segmented into 16 longitudinal strips 14 with a 2 mm gap between strips in substantially the same position in an MRI system 12 as the conventional shield 10. The adjacent strips 16 extend along the main magnetic field in MRI system 12 and are bridged together with 1000 pF capacitors 18 to make RF shield 14 essentially continuous for RF.

FIG. 2 illustrates sound pressure level sensors 20 attached near the left and right ears of a head-shaped anthropomorphic phantom 22 filled with tissue equivalent solution (only the right ear sensor 20 is visible). Tests with head phantom 22 demonstrate that the novel RF shield 14 reduces acoustic noise by up to 10 dB in a 7T MRI system. Notably, acoustic noise reduction as described in this patent specification can be achieved without significant compromising the transmit B1 field compared to the conventional double-layered slotted shield 10. The new type of RF shielding described in this patent specification substantially reduces acoustic noise while simultaneously improving the RF transmit efficiency.

Acoustic noise measurements were taken on an 8-channel transmit 64-channel receive head coil. Results are compared to a “reference” RF shield 10 that is a conventional two-layered design comprising 34 longitudinal strips in each layer with a strip width 32 mm and copper thickness 18 um in a typical position in MRI system 12.

It was established, as described further below, that a very significant source of acoustic noise beyond a cut-off frequency in the kHz range was due to eddy currents induced in a conventional RF shield rather than by vibrations of the gradient coils in an MRI system.

Sound pressure levels were measured using dedicated sensors 20 connected to a front-end and software (Bruël & Kjaer, Naerum, Denmark). To get a transfer function versus frequency, measurements were first performed with constant amplitude (G=2 mT/m) and linear frequency sweeps between 0 and 3.2 kHz in 2 min, for each gradient axis individually. Second, because the spectrum of MR sequences can be widespread, sound levels were also measured with EPI sequences at different echo spacings (0.56, 0.61, 0.66, 0.71, 0.76, 0.81, 0.96 and 1.01 ms) at constant gradient amplitude (76 mT/m), bandwidth and with the 3 readout axes (X, Y, Z) separately. The measurements were repeated at both ear locations of phantom 22. To verify that RF shield 14 was not detrimental to the transmit efficiency, an Actual Flip angle Imaging (AFI) sequence was implemented to measure the B1₊ field in circularly polarized (CP) mode.

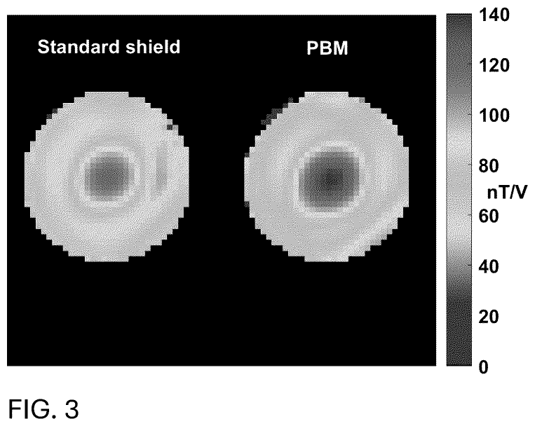

FIG. 3 shows B1 maps acquired in CP (Circularly Polarized) mode that demonstrate improved transmit performance with RF shield 14 (labelled PBM in FIG. 3) relative to a standard RF shield 10 (labelled Standard shield in FIG. 3). Shield 14 allows wider strips 16 to be used without increasing eddy currents, for a rough gain of 15% in B₁⁺ between the two shield configurations, according to some embodiments. The higher transmit efficiency increases the RF transmit energy to improve excitation of spins in the image volume to thereby raise SNR and improve image quality. The B1 maps in FIG. 3 were measured by driving the coil in CP mode, using the same reference voltage. The wider strips of the PBM shield 14 improved the overall transmit efficiency, yielding a gain in the center of about 15%.

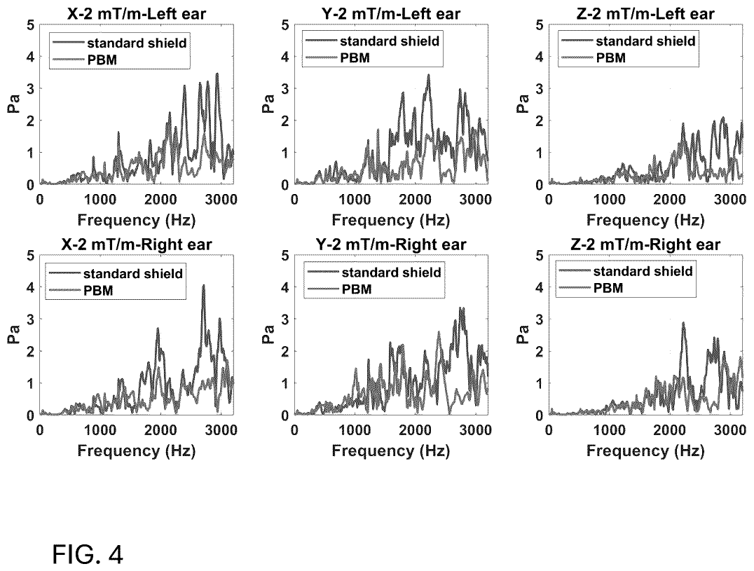

FIG. 4 shows acoustic transfer functions (in linear scale) demonstrating substantial noise reduction when using RF shield 14 relative to a standard RF shield 10 for all three gradient axes XYZ using an example of RF shield 14 providing up to 10 dB noise reduction, with measurements performed on each axis separately at constant gradient amplitude of 2 mT/m, according to some embodiments. Although the gain versus frequency is not systematic, the general trend is a drop of acoustic energy above 1.5 kHz, with some peaks even reduced sometimes by 10 dB. The three panels in the first row show results for the left ear sensor 20 for each of the XYZ axes, and the three panels in the second row show like results for sensor 20 at the right ear of phantom 22. The blue trace is for RF shield 10 (labelled standard shield) and the red trace is for RF shield 14 (labelled PBM).

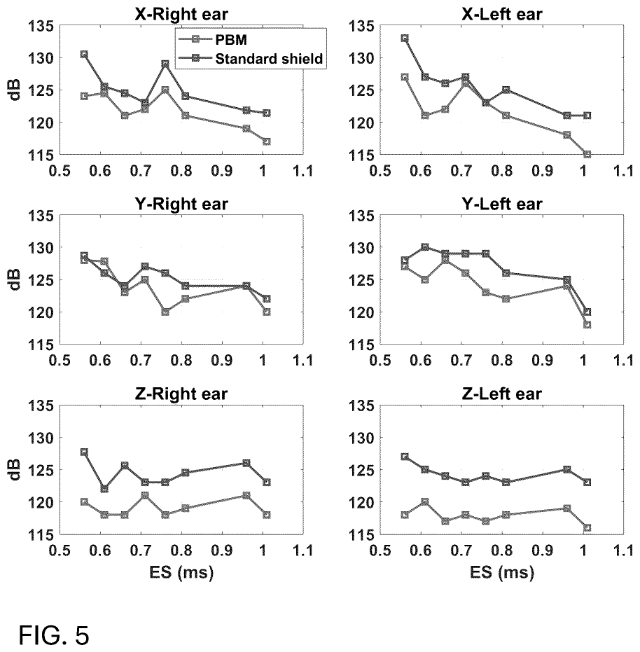

FIG. 5 shows a comparison of acoustic noise level in EPI acquisitions at different echo spacings, with RF shield 14, demonstrated almost systematically lower noise levels relative to using conventional shield 10. The vertical axis indicates sound pressure measured at sensors 20. Except for ES=0.61 ms, where the PBM shield 14 made it worse by 2 dB at the right ear and with the Y axis, the gains are systematic and vary between 0 and 9 dB. These results establish that current loops are disturbed and limited by segmenting and meshing the conductive surfaces, consequently minimising acoustic noise with RF shield 14.

The characteristics of RF shield 14 specified above are only examples and can vary as needed or desired for different applications and different MRI systems. For example, strips 16 of RF shield 14 can vary in number, spacing, length, width, thickness, and material so long as they fit in the space available for RF shielding an MRI system and reduce eddy currents and acoustic noise relative to a conventional RF shield 10 and preferably increase transfer of RF energy from the RF transmit coils to the imaging volume. As an example, if a mesh is used, the mesh count can be greater or lesser. As non-limiting examples: the number of strips 16 in RF shield 14 can be in the range 10-100 or more; the widths of strips 16 can be 1-200 mm or more; the spacing between adjacent strips 16 can be 1-200 mm; the axial length of strips 16 can be 100-1000 mm (limited of course by the available axial space in the magnet); the mesh count can be 10-1000 the thickness of strips 16 can be from a few microns to 2 cm; and the material of strips 16 can be other than PBM, such as a copper mesh for example, or a solid or perforated sheet of electrically conductive material. RF shield 14 can comprise a single layer of strips 16 or a double layer of strips 16 or can use more layers. Capacitors 18 can have different pF values and can bridge strips 18 in a different pattern, for example a capacitor 18 can bridge strips 16 that are not adjacent.

The results discussed above demonstrate that acoustic noise reduction can be achieved through different RF shield properties, without compromising the transmit B1 field. By identifying eddy-currents in the RF shield as a significant if not the main cause of acoustic noise, at least in the 7T system that was tested, these results pave the way for more improvements. While a specific example of RF shield 14 was tested, this patent specification contemplates using a shield 14 with smaller segments 16 along the axial and longitudinal directions. Overlapping the adjacent segments can further reduce the eddy currents and acoustic noise.

The discovery that eddy currents in an RF shield can be a significant if not a primary source of acoustic noise in high field MRI system such as a 7T Siemens scanner involved an investigation of acoustic noise sources described below. Motivation for this investigation was that such acoustic noise can be a severe physiological barrier for MRI acquisitions at high field. In this investigation, vibration and acoustic noise measurements were performed. Sponge window seal materials were stuck on the RF coil of the scanner to attempt to alter the vibrations of the RF coil and decrease acoustic noise. The experimental data is consistent with noise induced by eddy-currents on the conventional RF shield 10 (FIG. 1). Altering vibrations of the RF shield 10 allowed decreasing sound level by up to 10 dB at some EPI echo-spacings.

Higher main magnetic field, gradient strength and slew rates increase vibrations and acoustic noise according to the Laplace's force. The NexGen 7T scanner (Siemens) on which measurements were performed is equipped with the Impulse head gradient coil which yields unprecedented capabilities for high-resolution brain imaging. Despite successful in vivo acquisitions, high acoustic noise is detrimental to volunteer's comfort and may become in the future one of the most severe obstacles for MRI operation at higher fields. Although acoustic modes can play a role, acoustic noise in MRI is often attributed to the strong vibrations of the gradient coil. Foam rings can be placed around the boreliner to attenuate sound, after which very few ways seem easily available to MR users for more soundproofing, besides usual hearing protection or sequences avoiding mechanical resonances.

The values of bridging capacitors 16 preferably should be such that the capacitors present a low impedance that essentially shorts the strips to each other at the RF frequencies of operation of the MRI system. Two capacitors 18 bridge two adjacent strips 16 in the example of FIG. 1B, but more than two capacitors can be used to bridge two adjacent strips. The example of FIG. 1B shows strips 16 as continuous in the Z-direction (the long axis of magnet 12a) but according to some embodiments each strip 16 can be segmented in the Z-direction such that each of the illustrated strips 16 comprises two or more sub-strips that are adjacent to each other or are slightly spaced from each other in the Z-direction. In such a case, sub-strips that are adjacent to each other in the Z-direction can be bridged with respective capacitors like capacitors 18 to essentially short adjacent sub-strips to each other at the operating RF frequencies.

An investigation of origins of acoustic noise with vibration and sound pressure level (SPL) measurements on the Siemens NexGen scanner unexpectedly established that most of the noise originates from the eddy-currents induced in the RF shield.

Measurements were performed on the 7T NexGen Siemens scanner equipped with the Impulse head gradient (max gradient strength=200 mT/m/ms, max slew rate=900 mT/m/ms) and a 8Tx-64Rx head coil¹. SPL measurements (Optoacoustics, Mazor, Isreal) were first performed with continuous wave sinusoidal gradient waveforms at constant frequency and amplitude (5 mT/m), for each gradient axis, in 25 Hz steps, with and without the RF coil 10 and at the right ear location of an anthropomorphic phantom 22. Vibration measurements were performed on the external surface of the RF coil and on the patient table using mono axial accelerometers (B&K, Naerum, Denmark) with gradient sweeps over the 0-3 kHz range in 2 min and at 5 mT/m amplitude. To ascertain the possible influence of mechanical coupling between the patient table and the RF coil, different supporting materials between the two were tried: soft Sylodyn ND pads (Getzner, Bürs, Austria) and hard wooden wedges. Sponge window seals were finally stuck around the RF coil to aim at disturbing its mechanical vibration modes by making contact with the boreliner. SPL measurements were repeated in this configuration in vivo with EPI, at several echo-spacings (ES=0.56, 0.61, 0.66, 0.71, 0.76, 0.81, 0.96, 1.01 ms), at constant gradient amplitude (76 mT/m) and bandwidth, with the acoustic sensor placed at the left and right ears of the volunteer.

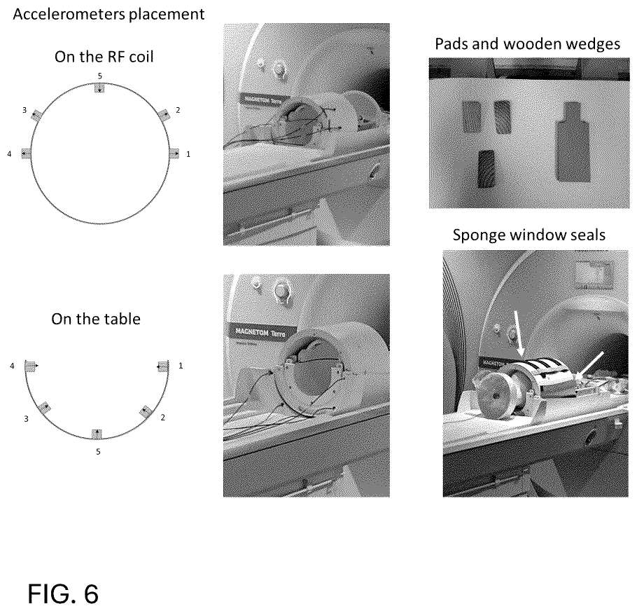

FIG. 6 illustrates a setup of accelerometers placed on an external surface of an RF coil (top left) or on a table (bottom left) to measure vibrations orthogonal to the surface. Different support materials and sponge window seals are shown and labeled on the right.

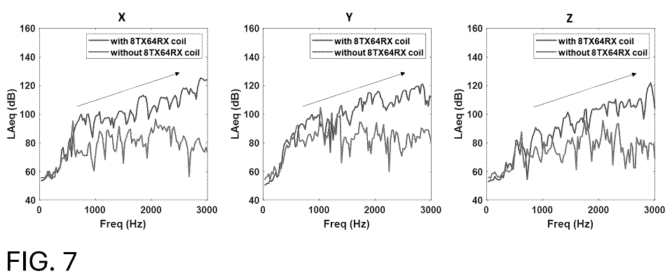

FIG. 7 illustrates SPL measurements performed at the right ear of an anthropomorphic phantom with and without the identified RF coil, versus frequency and at 5 mT/m; in the presence of the RF coil. An increasing trend of acoustic noise versus frequency, consistent with eddy-currents, is visible. The SPLs measured with and without the RF coil differ significantly, showing an increasing trend towards higher frequencies in the presence of the RF coil, which is consistent with eddy-currents (Faraday's law) induced in the shield 10 of the RF coil.

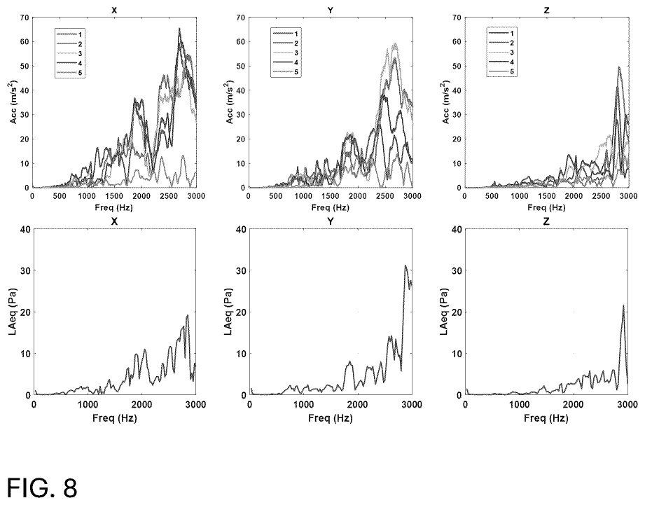

FIG. 8 illustrates vibration measurements (top) on the RF coil and acoustic noise levels (down, linear scale) versus frequency for respective gradient axes. Different colors on the top, labeled 1-4, correspond to different sensors. Reasonable correlation between the two can be observed, suggesting together with other data that most of the SPL originales from the vibration of the RF coil. The vibration of the table also was considerably lower without the RF coil than with the RF coil (data not shown).

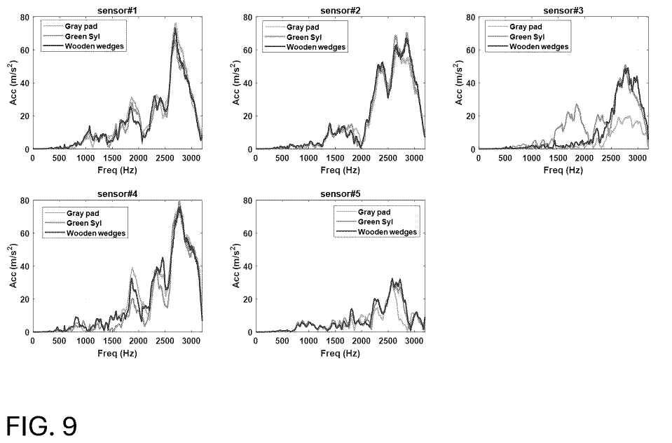

FIG. 9 illustrates vibration measurements on the RF coil versus frequency for the X gradient axis and with different supporting materials (the labeled pads and wooden wedges). Little differences can be observed. The differences on sensor #3 are believed due to different heights of the coil affecting the contact of the accelerometer with the boreliner. The different configurations made little difference, indicating that mechanical coupling between the bed and the RF coil, and thus gradient coil vibration, is likely not responsible for most of the acoustic noise. The data of sensor 3 revealed some differences which were attributed to the corresponding contact levels between the accelerometer and the boreliner, thereby indicating that mechanical vibrations, could be altered this way. Following this observation, the adhesive sponge window seals were placed to attempt establishing contact between the RF coil and the boreliner and disturb the vibrations. Acoustic measurements were performed in vivo in these conditions, with EPI and at various ES.

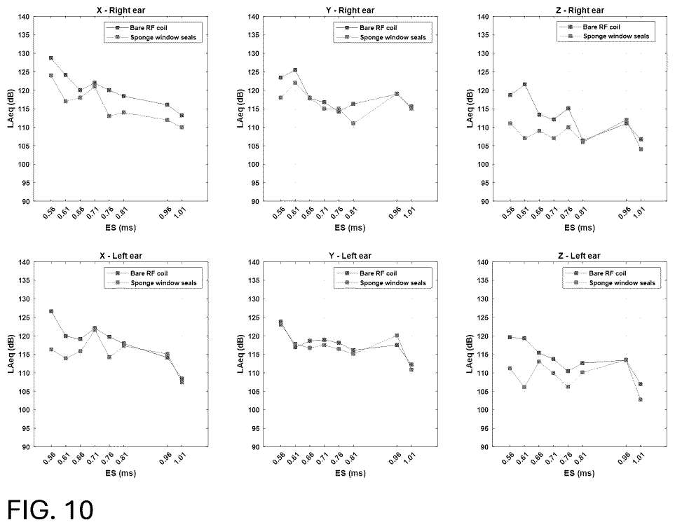

FIG. 10 illustrates SPL measurements performed at the right (top) and left (bottom) ear of a volunteer with and without sponge window seals. Although there is not a systematic gain, up to 10 dB improvement could be observed at short ES.

The tests with the setup of FIG. 6 indicate that eddy-current induced vibrations in the RF coil are an important factor responsible for acoustic noise. Alterations of the mechanical vibrations of the RF coil were achieved by providing a mechanical bridge with the boreliner and allowed decreasing SPL by up to 10 dB. Given the simplicity of this approach to affect the vibrations of the RF coil, the results pave the way for more investigations to reduce further the acoustic noise in MRI.

Although the foregoing has been described in some detail for purposes of clarity, it will be apparent that certain changes and modifications may be made without departing from the principles thereof. There can be many alternative ways of implementing both the processes and apparatuses described herein. Accordingly, the present embodiments are to be considered as illustrative and not restrictive, and the body of work described herein is not to be limited to the details given herein, which may be modified within the scope and equivalents of the appended claims.

The following references are hereby incorporated by reference in this patent application:
1. D A Feinberg et al., Nature Methods (December 2023)
2. Lee B J et al., MRM 79 (2018)
3. Shajan G et al., ISMRM 2022
4. Feinberg et al. In proceedings of the 2021 ISMRM and SMRT annual meeting, p 0562.
5. Winkler et al., Magn Reson in Med 2017; 78:1635-1645.

1. A Magnetic Resonance Imaging (MRI) system (12) configured to reduce acoustic noise when imaging a patient and increase transmit efficiency, comprising:
a magnet (12a) configured to provide a main magnetic field, gradient coils (12b) configured to selectively provide varying gradient magnetic fields, and a radio frequency (RF) transmit coil (12c) configured to selectively transmit RF pulses to an imaging volume; and
an RF shield (14) adjacent the RF transmit coil and comprising longitudinal strips (16) of an electrically conductive material and capacitors (18) bridging the strips, configured to reduce acoustic noise and to increase transmit efficiency of RF energy from the RF transmit coil to the imaging volume.

2. The MRI system of claim 1, in which the strips comprise a Phosphor Bronze Mesh (PBM).

3. The MRI system of claim 1, in which the strips are spaced from each other.

4. The MRI system of claim 1, in which the capacitors bridge adjacent strips and provide low impedance paths.

5. The MRI system of claim 1, in which the RF shield reduces eddy currents compared with an RF shield of overlapping strips of conductive material.

6. The MRI system of claim 1, in which the RF shield increases transmission of RF energy from the RF transmit coils to the imaging volume compared with an RF shield of overlapping strips of conductive material.

7. The MRI system of claim 1, in which the strips are elongated along the main magnetic field.

8. The MRI system of claim 1, in which the RF shield is a single layer of said strips.

9. The MRI system of claim 1, in which the strips are a Phosphor Bronze Mesh (PBM) with 380 mesh count per inch.

10. The MRI system of claim 1, in which the RF shield comprises 16 strips that are 2 mm apart and adjacent strips are bridged with 1000 pF capacitors.

11. The MRI system of claim 1, in which the strips (16) are spaced from each other to provide unobstructed gaps for passage of RF energy from said RF transmit coils.

12. A Magnetic Resonance Imaging (MRI) system comprising:
a magnet (12a) configured to provide a main magnetic field, gradient coils (12b) configured to provide gradient magnetic fields, and a radio frequency (RF) transmit coil (12c) configured to transmit RF pulses to an imaging volume; and
an RF shield (14) that is adjacent the RF transmit coil and comprises longitudinal strips (16) of an electrically conductive material and capacitors (18) bridging the strips;
wherein the strips are spaced from each other to leave gaps between adjacent strips facilitating increased transfer of RF energy from said RF transmit coils to said imaging volume compared with RF screens with overlapping strips of electrically conductive material.

13. The MRI system of claim 12, in which the RF shield comprises at least 10 strips.

14. The MRI system of claim 12, in which the strips are spaced from each other by at least 1 mm.

15. The MRI system of claim 12, in which the strips are in a single layer surrounding the imaging volume.

16. The MRI system of claim 12, in which the RF shield surrounds the RF transmit coils and the imaging volume.

17. A Magnetic Resonance Imaging (MRI) method comprising:
transmitting radio frequency (RF) energy from RF transmit coils to an imaging volume that is selectively subjected to a main magnetic field and time-varying gradient magnetic fields; and
shielding with an RF shied that is adjacent the RF transmit coils and reduces acoustic noise with strips of electrically conductive material that surround the imaging volume, are elongated along the main magnetic field and are spaced from each in a circumferential direction around the imaging volume, and are bridged with capacitors.

18. The method of claim 17, further including increasing transmission of RF energy from the RF transmit coils with said spaced-apart strips compared with an RF shield of overlapping strips of conductive material.

19. The method of claim 17, in which said shielding comprises arranging the strips in a single layer surrounding the imaging volume.

20. The method of claim 17, in which said shielding comprises arranging at least 10 of said strips uniformly spaced from each other around the imaging volume.
